(19) United States
(12) Patent Application Publication
(10) Pub. No.: US 2018/0064368 A1
(43) Pub. Date: Mar. 8, 2018

(54) Smartphone Based Peak Flow Meter

(71) Applicant: Ethan Wesley Chen, Yorba Linda, CA (US)

(72) Inventor: Ethan Wesley Chen, Yorba Linda, CA (US)

(21) Appl. No.: 15/256,025

(22) Filed: Sep. 2, 2016

(51) Int. Cl. A61B 5/087; A61B 5/097; A61B 5/09; A61B 5/00

(52) U.S. Cl. CPC A61B 5/0871; A61B 5/097; A61B 5/09; A61B 5/0022; A61B 5/6898; A61B 5/7278; A61B 5/0873

(57) ABSTRACT

Peak flow meters are often used by asthma patients to monitor their physical condition. It is imperative that a peak flow meter is conducive to patient usage and recording. A peak flow meter with a tube and rotary fin for use with a mobile device is described. The peak flow meter receives air from the patient through the tube which spins the rotary fin. The peak flow meter physically attaches to a mobile device. The rotation of the rotary fin interrupts the light going into the camera or light sensor of the mobile device. The rate of the interruption is converted to airflow measurement that is displayed to the user, saved to the mobile device, and uploaded to cloud storage.

### BACKGROUND OF THE INVENTION

### (1) Field of the Invention

This invention relates generally to the field of peak flow meters. Peak flow meters are used to measure a patient's peak expiratory flow rate which correlates to the presence of obstruction usually in the form of inflammation or swelling in the patient's airways.

### (2) Description of the Related Art

Prior art consists of portable peak flow meters both mechanical and digital based.

Mechanical peak flow meters operate through the use of a spring tab and a printed scale in order to measure maximum air flow rate (U.S. Pat. No. 5,627,324 A, 1997). The tab is driven by air flow provided by the user and then the user reads the value from the scale, similar to a ruler. The mechanical peak flow meter does not have any function by which a user can save their previous results. Furthermore, the size of most mechanical peak flow meters are prohibitive to regular usage. They are too large to carry around on one's person conveniently. Though prior art describes a miniature mechanical peak flow meter, the lack of automatic data registration is still a problem (U.S. Pat. No. 5,613,497 A, 1997).

Digital peak flow meters address the issue of manually recording the readings obtained from using a peak flow meter. Some peak flow meters are able to store readings on its own internal memory on the device (U.S. Pat. No. 5,816,246 A, 1998). Another function is to export readings to a personal computer (U.S. Pat. No. 5,518,002 A, 1996). Though digital peak flow meters can implement software that automatically collects patient data, cost can be prohibitive to usage and lead to low compliance. Prior art does describe a personal lung function monitoring device that can perform a number of lung function tests, including peak flow, that connects to a mobile device (USA Patent No. WO2013188458 A2, 2913). However this device requires a physical measuring device along with a microcontroller. The presence of a microcontroller in the device can be cost-prohibitive and raises the complexity of the device. Furthermore, a microcontroller necessitates that the device contains a charged battery in order to operate alongside a working mobile device. Thus the need exists for a low cost, miniature digital peak flow meter.

### BRIEF SUMMARY OF THE INVENTION

As mobile devices, particularly smartphones, are becoming more and more common and widely accessible, there exists the capability to leverage the sensors on these mobile devices, in particular the camera or an equivalent light sensor, to use in conjunction with a small plastic device to construct a low cost digital peak flow meter. This also allows the patient data to be recorded directly to the mobile device which can then be readily stored and communicated to a physician or medical professional.

Mobile devices, such as smartphones, are now more and more commonly used in point-of-care diagnostic testing in the field, particularly in areas with poor access to health care services. In third world countries, smartphones are increasingly being used as a focal point for portable medicine. Examples of medical uses for smartphones include pulse oximetry, endoscopes, otoscopes, ophthalmoscopes, and many others (USA Patent No. US20140159912 A1, 2014) (USA Patent No. WO2016019235 A1, 2016) (USA Patent No. US20120245422 A1, 2012) (USA Patent No. US 20120320340 A1, 2012). These smartphone compatible devices are attractive to use in the field because of their portability and low cost. The invent of a low cost peak flow meter attachment to a smartphone will add to the current portfolio of smartphone related medical devices and allow for diagnosis and monitoring of respiratory diseases.

### BRIEF DESCRIPTION OF THE SEVERAL VIEWS OF THE DRAWING

FIG. 1 is a close up view of the peak flow meter device attached to the corner of a mobile device.

FIG. 2 is a close-up view (hidden lines not shown) of the device while attached to a mobile device.

FIG. 3 shows a zoomed out view of the device attached to a mobile device.

FIG. 4 shows a view of the device only.

FIG. 5 shows a view of the turbine and encoder only placed on the camera/light sensor of a mobile device.

FIG. 6 shows a top down view of FIG. 5.

FIG. 7 shows a flow chart of the method of converting rotation of an encoder to air flow rate on a mobile device.

### DETAILED DESCRIPTION OF THE INVENTION

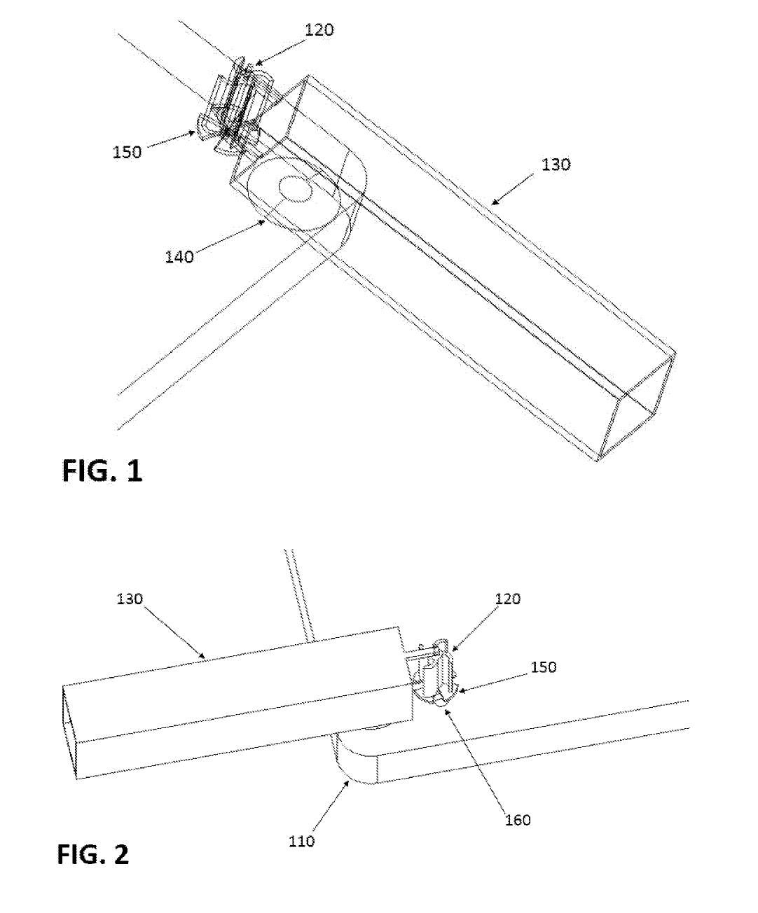

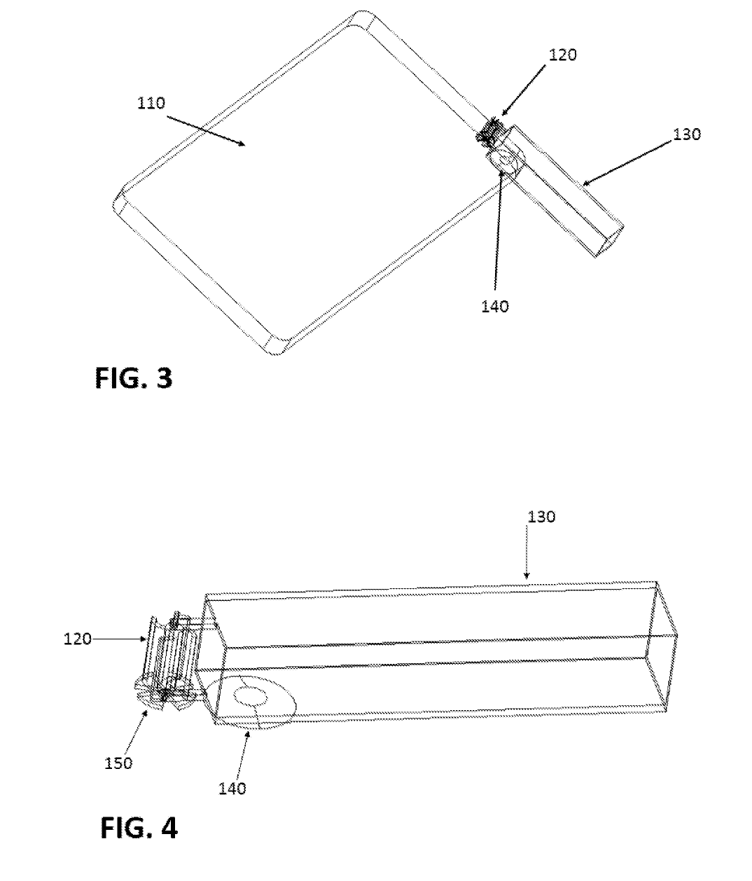

FIGS. 1-3 show a miniature peak flow meter that uses the camera or light sensor of a mobile device, such as a smartphone, in order to measure air flow rate. The device itself comprises of a small hollow rectangular tube 130 with a rotational turbine 120 and encoder wheel, a wheel with cut-in slots, 150 attached at the end. There also exists a suction cup 140 on the bottom of the tube that can be used to secure the peak flow meter in place on the mobile device. FIG. 3 shows the peak flow meter installed on a mobile device 110. FIGS. 1 and 2 shows a magnified view of FIG. 3 focusing on the peak flow meter itself.

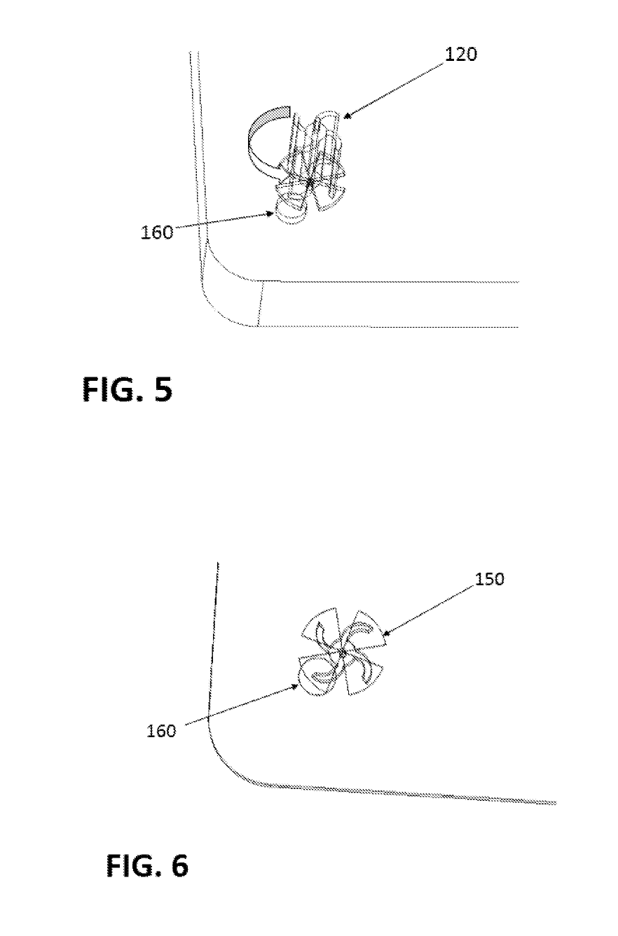

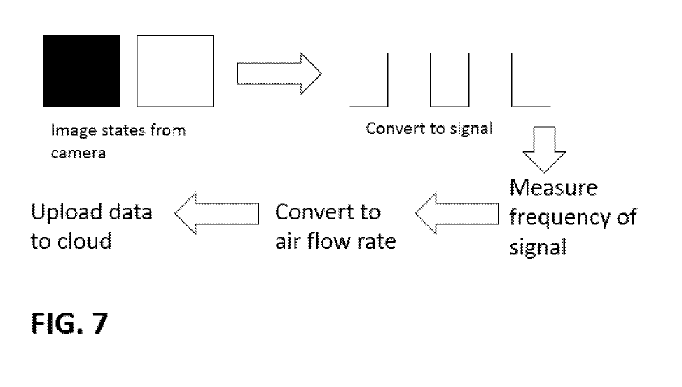

The peak flow meter is attached to the mobile device 110 through the usage of the attached suction cup 140. The encoder wheel 150 needs to be aligned with the camera/light sensor of the mobile device. The rotational turbine 120 is connected with the encoder wheel 150 in such a way that rotation of the turbine 120 as a result of blown air also rotates the encoder wheel 150. The rotation of the encoder wheel placed on top of the camera or light sensor will cause a variation in the brightness of the image seen by the camera/light sensor. FIG. 5 shows the placement of the encoder and turbine 120 over the camera/light sensor of the mobile device 160. FIG. 6 shows a view from the top of the same setup. The encoder wheel 150 can be clearly seen to block light from entering the camera/light sensor 160. This variation of light follows the pattern of a binary on/off or light/dark state and the rate of this pattern can then be used to calculate the rotational speed of the turbine and thus correlated to air flow speed. The light and dark states are calculated through the usage of software that utilizes image processing to take an average of the intensity of all pixels in each frame of the video collected by the camera/light sensor and comparing the result to a threshold value. The light and dark states are then converted to a wave signal of which the frequency is measured and converted to air flow rate. In order words, the rotational speed of the turbine is measured using the mobile device's camera/light sensor and then correlated to air flow rate. The patient air flow rate data is then stored in the mobile device's local data storage as well as uploaded to the cloud for access by a physician. The process is summarized on FIG. 7.

A summary of a typical order of usage follows. The user installs the peak flow meter by attaching the suction cup to the mobile device with the encoder wheel aligned with the camera/light sensor. Then the user opens the custom software, places their lips on the open end of the tube 130 and forcefully exhales. The software will then calculate the rotational speed of the turbine 120 and calculate maximum air flow rate.

### REFERENCES

Bing Yu, V. K. (2016). USA Patent No. WO2016019235 A1.
Cristina E. Davis, J. D. (2913). USA Patent No. WO2013188458 A2.
Debush, G. (1997). U.S. Pat. No. 5,613,497 A.
Fraden, J. (2014). USA Patent No. US20140159912 A1.
Hasburn, W. M. (2012). USA Patent No. US20120245422 A1.
James L. Wolf, D. V. (1996). U.S. Pat. No. 5,518,002 A.
Mirza, M. Z. (1998). U.S. Pat. No. 5,816,246 A.
Shene, W. R. (1997). U.S. Pat. No. 5,627,324 A.
Wyche T. Coleman, I. (2012). USA Patent No. US 20120320340 A1.

1. A peak flow meter that comprises:
a. a tube with an opening for receiving air flow
b. a rotary turbine
c. an encoder at the bottom of the rotary turbine that interrupts the light entering the camera of the mobile device
d. a suction cup or physical interface, such as a clip or clamp, to attach to a mobile device

2. The peak flow meter of claim 1, wherein the mobile device is a smartphone

3. A method of measuring peak air flow using a mobile device that comprises of the steps:
a. The usage of a camera or light sensor on said mobile device in order to measure air flow rate through the usage of a physical rotary turbine and encoder mounted on said mobile device.
b. A mobile application/software that measures the air flow rate through the light and dark states captured by the device's camera/light sensor. The frequency of the signal is then converted by the application/software to air flow rate.
c. The said mobile application/software that uploads the measurements to the cloud storage for distribution, reviewing and tracking.
